(19) United States
(12) Patent Application Publication
(10) Pub. No.: US 2007/0041987 A1
(43) Pub. Date: Feb. 22, 2007

(54) Fragments or polymers of albumin with tunable vascular residence time for use in therapeutic delivery and vaccine development

(76) Inventors: Daniel Carter, Huntsville, AL (US); Simon R. McKenzie, Huntsville, AL (US); Florian Ruker, Huntsville, AL (US)

Correspondence Address: STITES & HARBISON PLLC, 1199 NORTH FAIRFAX STREET, SUITE 900, ALEXANDRIA, VA, 22314, US

(21) Appl. No.: 10/549,809

(22) PCT Filed: Mar. 19, 2004
(86) PCT No.: PCT/US04/08696

§ 371 Date: Oct. 30, 2006

Related U.S. Application Data
(60) Provisional application No. 60/455,566, filed on Mar. 19, 2003.

(51) Int. Cl. A61K 39/00; C07H 21/04; C12P 21/06; C07K 14/765

(52) U.S. Cl. 424/185.1; 435/69.1; 435/320.1; 435/325; 530/363; 536/23.5

(57) ABSTRACT

An isolated fusion protein is provided which is a conjugate of a therapeutic polypeptide and an albumin fragment, such as a fragment including an individual domain or subdomain of albumin, or a polymer of albumin (e.g., dimers, trimers, etc.) and which is used to optimize the half-life of that therapeutic agent in the bloodstream in a tunable fashion based on the molecular weight of the fragment or polymer. Albumin fragments useful in the invention include fragments containing any of the individual domains and subdomains of human serum albumin, as well as fragments including specific combinations of binding regions or subdomains. The present invention thus provides fragments or polymers which will allow for optimizing half-lives of therapeutic polypeptides depending on their molecular weight, and this will optimizes protein and vaccine therapeutics to have desired half lives for their greatest effectiveness.

### CROSS-REFERENCE TO RELATED APPLICATIONS

The present application claims the benefit of U.S. provisional application 60/455,466, filed Mar. 19, 2003, and the specification of said provisional application is considered to be incorporated by reference into the present application as if set forth fully herein.

### FIELD OF THE INVENTION

The invention relates in general to conjugates of fragments and polymers of human serum albumin with therapeutic compounds, and in particular to a method of developing adjustable or tunable vasculature residence time of protein therapeutics, vaccines and other small molecules based on the conjugation of such materials with albumin fragments or polymers, such as specific albumin fragments from particular domains, subdomains or binding sites. The invention also relates to specific conjugates of albumin fragments to therapeutic agents, in particular whereby conjugation to these fragments will allow for specific half-lives in the circulation depending on the fragment size, and this is highly desirable because various protein therapeutics have different desired half-lives dependent on function and target tissue or organ.

### BACKGROUND OF THE INVENTION

It has been long been recognized that the ability of a therapeutic polypeptide or other compound to provide an important systemic effect in a human or animal patient is in large part tied to the particular half-life of the therapeutic agent in the blood stream or at target tissues. However, despite this knowledge, it has been a constant goal to increase or refine residence time of particular compounds or drugs so as to afford maximum benefit without decreasing the drug's effectiveness of incurring possibly dangerous side effects. These attempts have included invasive procedures such as drug delivery devices, such as described in published PCT application WO 00/41763, or liquid bioadhesive microemulsions or liposomic dispersions which may be used for transmucosal delivery of proteinic substances such as disclosed in U.S. Pat. No. 5,654,000, said patents incorporated herein by reference. However, in addition to being invasive, neither of these systems can be utilized in a safe and effective manner to increase the half-lives of important therapeutic compounds in the blood stream.

Similarly, previous attempts to utilize particular biomolecules in the extension of biological activity of a given therapeutic agent have not addressed the question of needed flexibility and the inherent differences among therapeutic agents and have thus not developed methods of maximizing the therapeutic effects of biomolecules of different half-lives and modes of activity. For example, previous efforts include the use of fusion polypeptides including human serum albumin and therapeutically active polypeptides, such as disclosed in U.S. Pat. Nos. 6,686,179, 6,165,470 and 5,876,969, and published US patent applications 2003/022308, 2003/0036170, 2003/0036170, 2003/0036170, and 2003/0036170, all of these patent references incorporated herein by reference. However, once again, these references focus on the use of albumin or its derivatives in fusing with particular polypeptides and do not provide an effective means for obtaining a flexible system which can tailor the desired increase or adjustment in residence time for a particular therapeutic compound based in the ideal criteria for such extension by virtue of the activity of the compound and its behavior in the bloodstream. It is thus still a highly desirable goal to obtain a system wherein the particular adjustments to the half-lives of therapeutic compounds can be carried out by use of a flexible system wherein a particular therapeutic polypeptide is conjugated to a particular biomolecule as needed to maximize the half-life and the therapeutic effect of that compound.

### SUMMARY OF THE INVENTION

Accordingly, it is an object of the present invention to provide a method of fusing proteins to specific albumin fragments or polymers of albumin (dimers, trimers, etc.) which will provide various residence times depending on their respective molecular weights.

It is another object of the present invention to provide specific conjugates made up of specific albumin fragments such as those fragments including the specific domains and subdomains of human serum albumin, and to utilize these conjugates in order to vary the half-lives of protein and other therapeutics conjugated to the albumin fragment so as to maximize the effect of the therapeutic polypeptide in a patient or to maximize the ability of a given antigen to generate suitable antibodies.

It is further an object of the present invention to provide therapeutic compositions and vaccines from fusion proteins and polypeptides with optimized vascular residence times so as to improve the likelihood of an immune response and provide prolonged therapeutic benefits from such vaccines and other therapeutic antigens.

It is yet another object of the present invention to provide recombinant fusion proteins conjugated to albumin fragments or dimers or trimers for use in vaccines, drug delivery, and other therapeutic methods involving proteins and peptide segments which can be fused to the albumin fragment or polymer without interfering with the therapeutic effect of the biologically active therapeutic polypeptide of the conjugate, and which have optimized residence times to maximize the therapeutic effects of the particular polypeptide.

These and other objects are provided by virtue of the present invention which comprises the fusion of a therapeutic agent to an appropriate albumin fragment, such as a fragment including an individual binding domain of albumin, or a polymer of albumin (e.g., dimers, trimers, etc.) so as to optimize the half-life of that therapeutic agent in the bloodstream in a tunable fashion based on the molecular weight of the fragment or polymer. Among the useful fragments of albumin which can be fused to therapeutic proteins and other agents include fragments containing any of the individual domains I, II and III of human serum albumin, as well as fragments including specific combinations of binding regions, including domains I-II, II-III, region IA-IB-IIA and IB-IIA-IIB. More specifically, some particular fragments useful in the present invention include HSA domain I-II, HSA domain IB-II, HSA domain I, HSA Domain I-IIA, HSM domain II and HSA Domain III. These fragments have been specifically disclosed in articles such as Dockal et al., J. Biol. Chem. 275(5):3042-3050 (2000) and Dockal et al., Protein Science 9:1455-1465 (2000), and these references are incorporated herein in their entirety and attached hereto. The human serum albumin molecule, the most abundant serum protein, is comprised of three homologous, primarily helical domains identified as domain I, domain II and domain III, each of which contains two subdomains, namely IA, IB, IIA, IIB and IIIA, IIIB. Unlike the whole albumin protein, these fragments generally have particular binding affinities and act as binding sites for different ligands. However, these fragments generally aid in albumin's ability to interact reversibly with a wide variety of endogenous and exogenous compounds so as to be useful as transport proteins. In general, ligands binding to albumin domain I are bulky heterocyclic anions with the charge situated in a central position of the molecule, and drugs that are bound to site II are generally aromatic carboxylic acids with an extended conformation and the negative charge located at one end of the molecule. Site I ligands include such compounds as coumarin anticoagulants, sulfonamides and salicylate. The present invention thus makes particular use of these fragments to target specific therapeutic polypeptides and to form fusion proteins with these polypeptides so as to give a therapeutic fusion polypeptide with an optimized half-life based on the molecular weight of the albumin fragment fused thereto.

In general, the present invention provides fragments which will allow for tunable or optimized half-lives of therapeutic agents in circulation depending on the fragment size, and this provides distinct advantages because various protein therapeutics have different desired half lives for the greatest effectiveness. Further, the invention also relates to the use of larger molecular weight polymeric albumin (dimers, trimers, etc.), which also exhibit different half lives in circulation, and which can also be used so as to provide a tunable residence time when fused with specific therapeutic agents.

These embodiments and other alternatives and modifications within the spirit and scope of the disclosed invention will become readily apparent to those skilled in the art from reading the present specification and/or the references cited herein, all of which are incorporated by reference.

### BRIEF DESCRIPTION OF THE DRAWING FIGURES

FIGS. 1A-1E are representations of five fragments of human serum albumin (HSA) useful in the present invention.

### DETAILED DESCRIPTION OF THE PREFERRED EMBODIMENTS

In accordance with the present invention, there are provided fusion polypeptides and proteins which comprise a fusion product between at least one therapeutic polypeptide and a fragment of human serum albumin, such as a fragment including an individual binding domain of albumin, or a polymer of albumin (e.g., dimers, trimers, etc.) so as to optimize the half-life of that therapeutic agent in the bloodstream in a tunable fashion based on the molecular weight of the fragment or polymer. By human serum albumin is meant the albumin protein found in human sera or any of the like proteins from other mammalian serum albumins, such as bovine serum albumin, baboon serum albumin, ovine serum albumin, etc., which has similar properties to human serum albumin and which can form fusion polypeptides in the same manner as human serum albumin. Similarly, by human serum albumin is also meant those albumin variants which encompass any albumin protein with a high plasma half-life which is obtained by modification (mutation, deletion and/or addition), by genetic engineering techniques, of a gene encoding a given isomorph of human serum albumin, as well as any macromolecule with a high plasma half-life obtained by in vitro modification of the protein encoded by such genes. Albumin being highly polymorphic, numerous natural variants have been identified and classified (see, e.g., Weitkamp et al., Ann. Hum. Genet. 37:219 (1973)).

By therapeutic polypeptide is meant those proteins and/or peptides which have a therapeutic effect on human or animal patients, and this can include those proteins, peptides, antibodies, fragments, enzymes, haptens, peptidoglycans or other molecules including amino acid sequences which can be linked to albumin or to a binding site, domain or subdomain, or combinations thereof, without disruption so that the fusion polypeptide when expressed will maintain the same or similar therapeutic activity as the unbound therapeutic polypeptide In one specific embodiment, the peptides possessing a therapeutic activity are not of human origin. For example, these may be peptides, or their derivatives, possessing properties which are potentially useful in the pathologies of the blood and interstitial compartments, such as hirudin, trigramine, antistatine, tick anticoagulant peptides (TAP), arietin, applagin and the like.

Additionally, in the fusion polypeptides of the present invention, the polypeptide having a therapeutic activity may be a polypeptide of human origin or a molecular variant. For example, this may be all or part of an enzyme, an enzyme inhibitor, an antigen, an antibody, a hormone, a factor involved in the control of coagulation, an interferon, a cytokine )the interleukins, but also their variants which are natural antagonists of their binding to the receptor(s), the SIS (small induced secreted) type cytokines and for example the macrophage inflammatory proteins (MIPs), and the like!, of a growth factor and/or of differentiation )and for example the transformant growth factors (TGFs), the blood cell differentiation factors (erythropoietin, M-CSF, G-CSF, GM-CSF and the like), insulin and the growth factors resembling it (IGFs), or alternatively cell permeability factors (VPFNEGF), and the like!, of a factor involved in the genesis/resorption of bone tissues (OIF and osteospontin for example), of a factor involved in cellular motility or migration )and for example autocrine motility factor (AMF), migration stimulating factor (MSF), or alternatively the scatter factor (scatter factor/hepatocyte growth factor)!, of a bactericidal or antifungal factor, of a chemotactic factor )and for example platelet factor 4 (PF4), or alternatively the monocyte chemoattracting peptides (MCP/MCAF) or neutrophil chemoattracting peptides (NCAF), and the like!, of a cytostatic factor (and for example the proteins which bind to galactosides), of a plasma (and for example von Willebrand factor, fibrinogen and the like) or interstitial (laminin, tenascin, vitronectin and the like) adhesive molecule or extracellular matrices, or alternatively any peptide sequence which is an antagonist or agonist of molecular and/or intercellular interactions involved in the pathologies of the circulatory and interstitial compartments and for example the formation of arterial and venous thrombi, cancerous metastases, tumour angiogenesis, inflammatory shock, autoimmune diseases, bone and osteoarticular pathologies and the like.

In the fusion polypeptides of the present invention, the active part of the polypeptides may consist for example of the polypeptide having a whole therapeutic activity, or of a structure derived therefrom, or alternatively of a non-natural polypeptide isolated from a peptide library. For the purposes of the present invention, a derived structure is understood to mean any polypeptide obtained by modification and preserving a therapeutic activity. Modification should be understood to mean any mutation, substitution, deletion, addition or modification of genetic and/or chemical nature. Such derivatives may be generated for various reasons, such as especially that of increasing the affinity of the molecule for its binding sites, that of improving its levels of production, that of increasing its resistance to proteases, that of increasing its therapeutic efficacy or alternatively of reducing its side effects, or that of conferring on it new biological properties. As an example, the therapeutic fusion polypeptides of the invention possess pharmacokinetic properties and a biological activity which can be used for the prevention or treatment of diseases, but more particularly are advantageous because they feature a conjugate that offers optimal residence time for the particular polypeptide therapeutic agent in the conjugate.

Particularly advantageous polypeptides of the invention are those in which the active part has its whole peptide structure or a structure derived by structural modification (mutation, substitution addition and/or deletion of one or more residues) and possessing a therapeutic activity. By therapeutic polypeptide in accordance with the invention, it is intended to mean those peptides and polypeptides with a therapeutic effect, and in addition small molecules that also have biological activity which like the peptides or polypeptides can have similar therapeutic activity. Among the suitable therapeutic polypeptides of the invention, there is preferably included molecules in which certain N- or O-glycosylation sites have been modified or suppressed, the molecules in which one or more residues have been substituted, or the molecules in which all the cysteine residues have been substituted. Other molecules useful in the invention may be obtained by deletion of regions not involved or not highly involved in the interaction with the binding sites considered, or expressing an undesirable activity, and molecules containing additional residues such as for example an N-terminal methionine and/or a signal for secretion and/or a joining peptide.

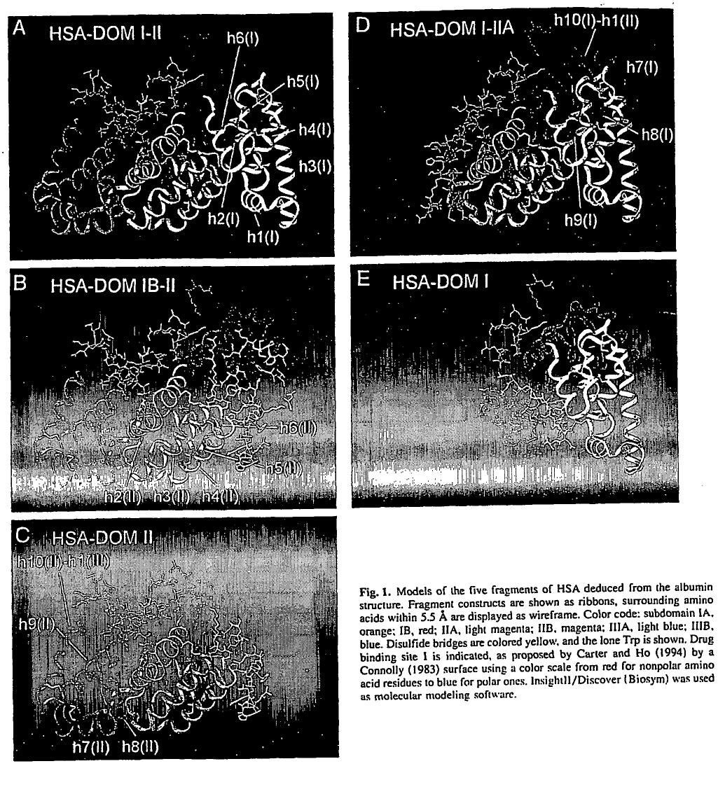

Among the useful fragments of albumin which can be fused to therapeutic polypeptides in accordance with the present invention include fragments containing any of the individual binding domains I, II and III of human serum albumin, as well as fragments including specific combinations of binding regions, including domains I-II, II-III, region IA-IB-IIA and IB-IIA-IIB. More specifically, some particular fragments useful in the present invention include HSA domain I-II, HSA domain IB-II, HSA domain I, HSA Domain I-IIA, HSA domain II and HSA Domain III. Representations of five of these albumin domains are shown in the drawing FIGS. 1A-1E.

Suitable albumin polymers useful in the invention will be any suitable polymer, e.g., dimers or trimers, which can bind to a therapeutic polypeptide and extend its half-live without a substantial effect on its therapeutic activity. As would be recognized by one of ordinary skill in the art, the number of albumin monomers in the polymer of the present invention is typically a low number since after a given number of albumin molecules, the molecule either becomes unstable or will be unsuitable for use as a fusion protein. Accordingly, the preferred albumin polymer in accordance with the invention is one that has from 2 to 5 albumin monomers.

In addition to having specific albumin fragments covering the binding domains and subdomains as set forth above, it is also possible to use polymeric forms of these albumin fragments. Once again, the number of fragments will be adjusted so as to optimize the half-life of a therapeutic polypeptide to which it is attached, but this number is variable and will depend on the conjugate and the nature of the target tissue or organ.

Human serum albumin is one of a series of serum albumins which are the major soluble proteins of the circulatory system and contribute to many vital physiological processes. Serum albumin generally comprises about 50% of the total blood component by dry weight, and as such is responsible for roughly 80% of the maintenance of colloid osmotic blood pressure and is chiefly responsible for controlling the physiological pH of blood. The albumins also play an extremely important role in the transport, distribution and metabolism of many endogenous and exogenous ligands in the human body, including a variety of chemically diverse molecules including fatty acids, amino acids, steroids, calcium, metals such as copper and zinc, and various pharmaceutical agents. The albumins are generally thought to facilitate transfer many of these ligands across organ-circulatory interfaces such as the liver, intestines, kidneys and the brain, and studies have suggested the existence of an albumin cell surface receptor. See, e.g., Schnitzer et al., P.N.A.S. 85:6773 (1988), incorporated herein by reference. The albumins are thus intimately involved in a wide range of circulatory and metabolic functions.

Human serum albumin (HSA) is a protein of about 66,500 kD protein and is comprised of 585 amino acids including at least 17 disulfide bridges. As with many of the albumins, human serum albumin plays an extremely important role in human physiology and is located in virtually every human tissue and bodily secretion. As indicated above, HSA has an outstanding ability to bind and transport and immense spectrum of ligands throughout the circulatory system including the long-chain fatty acids which are otherwise insoluble in circulating plasma. Certain details regarding the atomic structure and the binding affinities of albumin and the specific regions primarily responsible for those binding properties have previously been disclosed, e.g., in U.S. patent application Ser. No. 08/448,196, filed May 25, 1993, now U.S. Pat. No. 5,780,594 and U.S. patent application Ser. No. 08/984,176, filed Dec. 3, 1997, now U.S. Pat. No. 5,948,609, both of which are incorporated herein by reference.

Because of the vital role played by albumins, there are literally thousands of applications for serum albumin covering a wide range of physiological conditions. Similarly, because of the nature of the binding sites which act to bind a variety of ligands, it is thus ideally useful to utilize said fragments as conjugates with a variety of therapeutic polypeptides to form fusion proteins which have vascular residence times suitable for the given purpose of the therapeutic agent. As indicated above, the albumin fragments or polymers useful in the invention may be obtained from natural isolates, or preferably can be prepared through recombinant means so as to provide a safe and effective fusion protein which can be administered in the blood stream of a patient. Thus, the recombinant production of the albumin fragments or polymers can take place using any suitable conventional recombinant technology currently known in the field. For example, molecular cloning of the fusion protein of the invention can be carried out via expression in E. coli using nucleic acid coding for the particular albumin fragment or polymer utilized for a particular therapeutic polypeptide, and this can be cloned along with the nucleic acid coding for the particular polypeptide so that both are expressed. The resulting fusion peptide or protein may be isolated and/or purified, such as from a supernatant obtained from the E. coli culture, using appropriate chromatographic or other methods, such as Size Exclusive and Gel Filtration Ion Exchange chromatography. The protein or peptide may then be confirmed using conventional Western blot tests using suitable polyclonal and monoclonal antibodies.

Among the eukaryotic hosts which can be used within the framework of the present invention, there may be mentioned animal cells, yeasts or fungi. In particular, as regards yeasts, there may be mentioned yeasts of the genus Saccharomyces, Kluyveromyces, Pichia, Schwanniomyces, or Hansenula. As regards animal cells, there may be mentioned COS, CHO and C127 cells and the like. Among the fungi capable of being used in the present invention, there may be mentioned more particularly Aspergillus ssp, or Trichoderma ssp. As prokaryotic hosts, the use of bacteria such as Escherichia coli, or belonging to the genera Corynebacterium, Bacillus, or Streptomyces is preferred.

As set forth above, the nucleotide sequences which can be used within the framework of the present invention can be prepared in various ways. Generally, they are obtained by assembling, in reading phase, the sequences encoding each of the functional parts of the polypeptide. The latter may be isolated by the techniques of persons skilled in the art, and for example directly from cellular messenger RNAs (mRNAs), or by recloning from a complementary DNA (cDNA) library, or alternatively they may be completely synthetic nucleotide sequences. It is understood, furthermore, that the nucleotide sequences may also be subsequently modified, for example by the techniques of genetic engineering, in order to obtain derivatives or variants of the said sequences. More preferably, in the process of the invention, the nucleotide sequence is part of an expression cassette comprising a region for initiation of transcription (promoter region) permitting, in the host cells, the expression of the nucleotide sequence placed under its control and encoding the polypeptides of the invention. This region may come from promoter regions of genes which are highly expressed in the host cell used, the expression being constitutive or regulatable. As regards yeasts, it may be the promoter of the gene for phosphoglycerate kinase (PGK), glyceraldehyde-3-phosphate dehydrogenase (GPD), lactase (LAC4), enolases (ENO), alcohol dehydrogenases (ADH), and the like. As regards bacteria, it may be the promoter of the right-hand or left-hand genes from the lambda bacteriophage (P.sub.L, P.sub.R), or alternatively the promoters of the genes for the tryptophan (P.sub.trp) or lactose (P.sub.lac) operons. In addition, this control region can be modified, for example by in vitro mutagenesis, by the introduction of additional control elements or of synthetic sequences, or by deletions or substitutions of the original control elements. The expression cassette may also comprise a region for termination of transcription which is functional in the host envisaged, positioned immediately downstream of the nucleotide sequence encoding a polypeptide of the invention.

In a preferred mode, the polypeptides of the invention result from the expression, in a eukaryotic or prokaryotic host, of a nucleotide sequence and from the secretion of the product of expression of the said sequence into the culture medium. It is indeed particularly advantageous to be able to obtain, by the recombinant route, molecules directly in the culture medium. In this case, the nucleotide sequence encoding a polypeptide of the invention is preceded by a “leader” sequence (or signal sequence) directing the nascent polypeptide in the secretory pathways of the host used. This “leader” sequence may be the natural signal sequence of the biologically active polypeptide in the case where the latter is a naturally secreted protein, or that of the stabilizing structure, but it may also be any other functional “leader” sequence, or an artificial “leader” sequence. The choice of one or the other of these sequences is especially guided by the host used. Examples of functional signal sequences include those of the genes for the sexual pheromones or the “killer” toxins of yeasts. In addition to the expression cassette, one or several markers which make it possible to select the recombinant host may be added, such as for example the URA3 gene from the yeast S. cerevisiae, or genes conferring the resistance to antibiotics such as geneticin (G418) or to any other toxic compound such as certain metal ions.

The unit formed by the expression cassette and by the selectable marker can be introduced directly into the considered host cells, or previously inserted in a functional self-replicating vector. In the first case, sequences homologous to regions present in the genome of the host cells are preferably added to this unit; the said sequences then being positioned on each side of the expression cassette and of the selectable gene so as to increase the frequency of integration of the unit into the genome of the host by targeting the integration of the sequences by homologous recombination. In the case where the expression cassette is inserted in a replicative system, a preferred replication system for yeasts of the genus Kluyveromyces is derived from the plasmid pKD1 originally isolated from K. drosophilarum; a preferred replication system for yeasts of the genus Saccharomyces is derived from the 2.mu. plasmid from S. cerevisiae. Furthermore, this expression plasmid may contain all or part of the said replication systems, or may combine elements derived both from the plasmid pKD1 and the 2.mu. plasmid. In addition, the expression plasmids may be shuttle vectors between a bacterial host such as Escherichia coli and the chosen host cell. In this case, a replication origin and a selectable marker functioning in the bacterial host are required. It is also possible to position restriction sites surrounding the bacterial and unique sequences on the expression vector: this makes it possible to suppress these sequences by cutting and religation in vitro of the truncated vector before transformation of the host cells, which may result in an increase in the number of copies and in an increased stability of the expression plasmids in the said hosts

After construction of such vectors or expression cassette, the latter are introduced into the host cells selected according to the conventional techniques described in the literature. In this respect, any method permitting the introduction of a foreign DNA into a cell can be used. This may be especially transformation, electroporation, conjugation, or any other technique known to persons skilled in the art. As an example of yeast-type hosts, the various strains of Kluyveromyces used were transformed by treating the whole cells in the presence of lithium acetate and polyethylene glycol, according to the technique described by Ito et al. J. Bacteriol. 153:163 (1983). The transformation technique described by Durrens et al. Curr. Genet. 18:7 (1990) using ethylene glycol and dimethyl sulphoxide may also be used. It is also possible to transform the yeasts by electroporation, according to the method described by Karube et al. FEBS Letters 182:90 (1985). After selection of the transformed cells, the cells expressing the said polypeptides are inoculated and the recovery of the said polypeptides can be carried out, either during the cell growth for the “continuous” processes, or at the end of growth for the “batch” cultures. The polypeptides which are the subject of the present invention are then purified from the culture supernatant for their molecular, pharmacokinetic and biological characterization.

One preferred expression system for the polypeptides of the invention consists in using yeasts of the genus Kluyveromyces as host cell, transformed by certain vectors derived from the extrachromosomal replicon pKD1 originally isolated from K. marxianus var. drosophilarum. These yeasts, and in particular K. lactis and K. fragilis are generally capable of stably replicating the said vectors and possess, in addition, the advantage of being included in the list of G.R.A.S. (“Generally Recognized As Safe”) organisms. Favored yeasts are preferably industrial yeasts of the genus Kluyveromyces which are capable of stably replicating the said plasmids derived from the plasmid pKD1 and in which has been inserted a selectable marker as well as an expression cassette permitting the secretion, at high levels, of the polypeptides of the invention. The present invention thus relates to the nucleotide sequences encoding the fusion polypeptides described above, as well as the eukaryotic or prokaryotic recombinant cells comprising such sequences. The present invention also relates to the application, as medicinal products, of the fusion polypeptides according to the present invention. More particularly, the subject of the invention is any pharmaceutical composition comprising one or more fusion polypeptides or nucleotide sequences as described above. The nucleotide sequences can also be used in gene therapy.

In accordance with the invention, the fusion polypeptides of the invention may be prepared by conjugating a therapeutic polypeptide to an albumin fragment or polymer as defined above by any suitable manner known to those of ordinary skill in the art. This can be done by a chemical reaction to cross link them or otherwise bond these together in a suitable manner, e.g., covalent bonding. For example, in the preferred process, the therapeutically active molecules of the invention can be coupled either directly or via an artificial peptide to the albumin fragment or polymer. Furthermore, it may constitute the N-terminal end as well as the C-terminal end of the fusion polypeptide conjugate formed in accordance with the invention. In one preferred mode of the invention, the active part constitutes the C-terminal part of the fusion polypeptide. It is also understood that the biologically active part may be repetitive within the fused polypeptide of the invention.

Previously, it has been known to prepare artificial proteins which are biologically active and can be used pharmaceutically. However, it has not been previously known to use particular proteins, such as albumin, its fragments and polymeric forms, in a system by which the residence time of a particular therapeutic polypeptide is optimized so as to afford the maximum benefit of the therapeutic agent prior to the time wherein it solubilizes and/or becomes ineffective or even harmful. The present invention thus provides new conjugates which permit an optimal therapeutic exploitation of the biological properties of these polypeptides based on the adjustment and optimization of residence time by virtue of the albumin fragment or polymer it may be conjugated to. Moreover, since human serum albumin has previously been demonstrated to present the active structure to sites for interaction and provides a high plasma stability for the therapeutic polypeptides of the invention, the present invention can be utilized to stably adjust the residence time of that therapeutic in the patient and allow it to provide its biological activity for an optimized period. This will result in a more efficient use of the therapeutic polypeptide with less chance of undesirable side effects.

In the preferred method of the present invention, fragments of albumin of varying length and molecular weight would be linked by recombinant or other methods to a desired therapeutic protein, antigenic peptide or small molecule, for the purpose of tuning the vascular residence time. It is known that albumin and antibodies, possess the longest half life of all the circulating plasma proteins. In the special case of antibodies, it is known that a specified receptor-based amino acid sequence on the Fe protein of the antibody allows for an additional method of vascular retention. In a simplified model it appears that proteins in the plasma exhibit the following properties: 1) those smaller than albumin exhibit reduced vascular resident times; 2) those approaching albumin in molecular weight (66,500) have the greatest vascular residence times; and those greater than 66,500 shows reduced vascular residence. For example, prothrombin has a half life in circulation of 1.7 days (Halton, M. W., J. Lab. Clin. Med. 1995: Vol. 126-521-529 Recombinant rabbit Albumin fragments containing sequences 1-185, 1-377, and 378-584 were excreted rapidly in the urine and possess plasma life's of only 0.71 days (Sheffield, et., Thrombosis Research 99, 613-621 (2000). Recently, work by the same group has verified that disulphide-linked albumin dimers are cleared more rapidly than the parent molecule having a ½ life reduced by approximately 60% that of the monomer (McCurdy et al., J. Lab. Clin. Med. (2004) 143(2) 115-124). Although, surface charge has been implicated as one of the active processes in the retention of plasma proteins, clearly molecular weight is playing a predominant role with regard to albumin. In the practice of this invention and in order to obtain therapeutic proteins and other agents with the desired vascular residence times, peak vascular residence times should be achieved for those fusion proteins with molecular weights approaching albumin. In this case to obtain a maximum extension of the ½ life for a therapeutic polypeptide in accordance with the invention (e.g., as indicated above, peptides, polypeptides, proteins or similar small molecules) of 20,000 MW would require the construction of a recombinant fusion with an albumin fragment with a molecular weight of approximately 45,000. Therapeutic polypeptides of higher molecular would similarly be fused with albumin fragments or polymers in the same manner. Molecular weights greater than albumin will again reduce the ½ life. Recombinant albumin fusion products which involve polymers of albumin or polymers of albumin fragments can therefore also be used to tune the vascular residence times of therapeutic agents. Variations from this rule would be expected to occur on a case by case basis depending on among other factors, glycosylation of the therapeutic portion of the fusion product, or other factors, which induce catabolism and rapid clearance. Consequently, examining the vasculature residence times of various fragments when attempted to assess which fragment will result in an optimal fusion polypeptide can also be applied to obtained the desired result.

Accordingly, in one aspect of the preferred method of optimizing the half-life of a therapeutic polypeptide when internally administered to humans or animals, the present invention preferably comprises a process wherein a fusion polypeptide is formed between said therapeutic polypeptide and a fragment of human serum albumin (as set forth above) containing at least one domain or subdomain or combinations thereof by attaching said albumin fragment to said therapeutically active polypeptide in such a manner wherein the human serum albumin fragment optimizes the half-life of said therapeutically active polypeptide in the bloodstream depending on the molecular weight of the fragment. In this manner, the half-life of the therapeutic polypeptide may be extended when such extension is optimal, or may be decreased when such decrease is optimal for a given application. Similarly, in another aspect of the invention, a method of optimizing the half-life of a therapeutic polypeptide when internally administered to humans or animals is provided which comprises forming a fusion polypeptide between said therapeutic polypeptide and a polymer of human serum albumin as described above in such a manner wherein the human serum albumin polymer optimizes the half-life of said therapeutically active polypeptide in the bloodstream depending on the molecular weight of the polymer.

The fusion polypeptides of the present invention may thus be utilized to enhance the properties of a number of proteins and peptides which are administered internally for a therapeutic purpose. In particular, through linkage with albumin, albumin fragments, or the albumin polymers as described above, the therapeutic polypeptide of the invention will have its half life in plasma optimized, which in some cases will extend the half-life of the therapeutic agent so it can continue providing therapeutic benefits long after the non-fused protein or peptide would have been completed degraded in the bloodstream. In addition, the fusion polypeptides of the invention can be used in some cases to enhance the immunogenic properties of a particular antigen since it can extend that antigen's time in the blood stream and maximize the ability of that antigen to generate useful antibodies.

In accordance with the present invention, the invention also provides isolated antibodies and antisera which recognize the conjugates of albumin fragments or polymers with a therapeutic polypeptide as set forth above. These antibodies may be generated in any suitable way, such as through polyclonal or monoclonal means, but the particularly advantageous use of the present invention is the generation of antibodies in the patient when administered an immunogenic amount of the fusion polypeptide of the present invention.

Similarly, the present invention contemplates the use of the fusion polypeptides of the invention as vaccines, once again with optimized residence time so as to obtain the maximum immunogenic effect while at the same time minimizing any deleterious side effects caused by the presence of the bioactive agent. These fusion polypeptide vaccines by act in both active and passive immunization schemes against infections, but in the preferred embodiment will comprise active vaccines wherein an immunogenic amount of a therapeutic antigenic fusion polypeptide is administered to the patient in order to have the patient generate antibodies thereto, and by the proper selection of the albumin fragment or polymer as described above, this immunogenic time may be optimized based on the desired effect.

As would also be recognized by one skilled in this art, vaccines in accordance with the present invention may be packaged for administration in a number of suitable ways, such as by parenteral (i.e., intramuscular, intradermal or subcutaneous) administration or nasopharyngeal (i.e., intranasal) administration. One such mode is where the vaccine is injected intramuscularly, e.g., into the deltoid muscle, however, the particular mode of administration will depend on the nature of the infection to be dealt with and the condition of the patient. The vaccine is preferably combined with a pharmaceutically acceptable carrier to facilitate administration, and the carrier is usually water or a buffered saline, with or without a preservative. The vaccine may be lyophilized for resuspension at the time of administration or in solution.

The preferred dose for administration of the fusion polypeptide in accordance with the present invention is those amounts which will be effective in immunizing a patient, i.e., in having that patient develop antibodies against a general or specific condition. This amount is generally referred to as an “immunogenic amount”, and this amount will vary greatly depending on the nature of the antigen and of the immune system and the condition of the patient. Thus an “immunogenic amount” of fusion protein used in accordance with the active vaccines of the invention is intended to mean a nontoxic but sufficient amount of the antigenic agent such that the desired prophylactic or therapeutic generation of antibodies is produced. Accordingly, the exact amount of the immunogenic agent that is required will vary from subject to subject, depending on the species, age, and general condition of the subject, the severity of the condition being treated, the particular carrier or adjuvant being used and its mode of administration, and the like. Accordingly, the “immunogenic amount” of any particular fusion polypeptide composition will vary based on the particular circumstances, and an appropriate immunogenic amount may be determined in each case of application by one of ordinary skill in the art using only routine experimentation. The dose should be adjusted to suit the individual to whom the composition is administered and will vary with age, weight and metabolism of the individual. The compositions may additionally contain stabilizers or pharmaceutically acceptable preservatives, such as thimerosal (ethyl(2-mercaptobenzoate-S)mercury sodium salt) (Sigma Chemical Company, St. Louis, Mo.). Moreover, because of the conjugation with the albumin fragments polymers as set forth, the fusion polypeptides of the invention generally have increased stability and longer usable shelf life.

Accordingly, an active vaccine in accordance with the invention is provided wherein an immunogenic amount of an isolated-fusion polypeptide as described above is administered to a human or animal patient in need of such a vaccine. The vaccine may also comprise a suitable, pharmaceutically acceptable vehicle, excipient or carrier. In accordance with the invention, it is thus possible to link the albumin fragment or polymer with any protein, peptide or polypeptide that may be useful in generating important antibodies, and thus particular viral or polyviral proteins, e.g., envelope proteins or other proteins from such potentially highly pathogenic viruses such as AIDS, SARS, etc., may also be used in the fusion polypeptides in environments wherein they can be introduced so as to develop antibodies against the AIDS and/or SARS viruses. In addition to providing vaccines which may be protective against such potentially deadly diseases, such fusion polypeptides may also be utilized in research concerning these diseases, and may be useful in developing methods or drugs in addition to vaccines which can be effective against these diseases.

In addition to the modes described above, it is also possible to link or conjugate the albumin fragments or polymers with particular antibodies so as to provide passive vaccines for patients in need of such treatment. In this case, the preferred dose for administration of an antibody composition in accordance with the present invention is that amount will be effective in preventing of treating a particular infection or condition, and, as stated above, by selection of the appropriate albumin fragment or polymer, the residence time of the conjugated antibody may be optimized to afford the maximum benefit of its biological activity. This optimal time will vary greatly depending on the nature of the infection and the condition of a patient, and one skilled in the art will recognize that an “effective amount” of such a fused antibody used in accordance with the invention is intended to mean a nontoxic but sufficient amount of the antibody such that the desired prophylactic or therapeutic effect is produced. Accordingly, the exact amount of the antibody or a particular agent that is required will vary from subject to subject, depending on the species, age, and general condition of the subject, the severity of the condition being treated, the particular carrier or adjuvant being used and its mode of administration, and the like. Accordingly, the “effective amount” of any particular antibody composition will vary based on the particular circumstances, and an appropriate effective amount may be determined in each case of application by one of ordinary skill in the art using only routine experimentation. The dose should be adjusted to suit the individual to whom the composition is administered and will vary with age, weight and metabolism of the individual. The compositions may additionally contain stabilizers or pharmaceutically acceptable preservatives, such as thimerosal (ethyl(2-mercaptobenzoate-S)mercury sodium salt) (Sigma Chemical Company, St. Louis, Mo.).

In addition, the antibody compositions of the present invention and the vaccines as described above may also be administered with a suitable adjuvant in an amount effective to enhance the immunogenic response against the conjugate. For example, suitable adjuvants may include alum (aluminum phosphate or aluminum hydroxide), which is used widely in humans, and other adjuvants such as saponin and its purified component Quil A, Freund's complete adjuvant, and other adjuvants used in research and veterinary applications. Still other chemically defined preparations such as muramyl dipeptide, monophosphoryl lipid A, phospholipid conjugates such as those described by Goodman-Snitkoff et al. J. Immunol. 147:410-415 (1991) and incorporated by reference herein, encapsulation of the conjugate within a proteoliposome as described by Miller et al., J. Exp. Med. 176:1739-1744 (1992) and incorporated by reference herein, and encapsulation of the protein in lipid vesicles such as Novasome™ lipid vesicles (Micro Vescular Systems, Inc., Nashua, N.H.) may also be useful.

As would be recognized by one skilled in the art, the fusion polypeptides of the invention may also be formed into suitable pharmaceutical compositions for administration to a human or animal patient in order to treat or prevent infections, or to be used as therapeutic agents against other diseases or conditions. Pharmaceutical compositions containing the fusion polypeptides of the present invention as defined and described above may be formulated in combination with any suitable pharmaceutical vehicle, excipient or carrier that would commonly be used in this art, including such as saline, dextrose, water, glycerol, ethanol, other therapeutic compounds, and combinations thereof. As one skilled in this art would recognize, the particular vehicle, excipient or carrier used will vary depending on the patient and the patient's condition, and a variety of modes of administration would be suitable for the compositions of the invention, as would be recognized by one of ordinary skill in this art. Suitable methods of administration of any pharmaceutical composition disclosed in this application include, but are not limited to, topical, oral, anal, vaginal, intravenous, intraperitoneal, intramuscular, subcutaneous, intranasal and intradermal administration.

For topical administration, the composition is formulated in the form of an ointment, cream, gel, lotion, drops (such as eye drops and ear drops), or solution (such as mouthwash). Wound or surgical dressings, sutures and aerosols may be impregnated with the composition. The composition may contain conventional additives, such as preservatives, solvents to promote penetration, and emollients. Topical formulations may also contain conventional carriers such as cream or ointment bases, ethanol, or oleyl alcohol.

As set forth above, in accordance with the invention, the fusion polypeptides of the invention have a number of potential uses in both the area of vaccines and other pharmaceutical and therapeutic compositions, as well as in many other areas which can provide beneficial effects. For example, the fusion polypeptides of the invention can be used to assess or maximize drug delivery and provide drug profiles so as to assess organ/tissue specificity of certain drugs or other compounds.

In short, the fusion polypeptides of the present invention are preferably utilize in methods of optimizing residence times of therapeutic polypeptides and thus can be extremely useful in maximizing therapeutic or immunogenic effects of particular therapeutic polypeptides, particularly in those cases wherein optimization of enhancement of vascular residence time is desired.

The present invention will be more fully described with the aid of the following examples, which should be considered as illustrative and non-limiting.

### EXAMPLE

The following example is provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

In the preferred application, fragments of albumin of varying length and molecular weight would be linked by recombinant or other methods to a desired therapeutic protein, antigenic peptide or small molecule, for the purpose of tuning the vascular residence time. It is known that albumin and antibodies, possess the longest half life of all the circulating plasma proteins. In the special case of antibodies, it is known that a specified receptor-based amino acid sequence on the Fe protein of the antibody allows for an additional method of vascular retention. In a simplified model it appears that proteins in the plasma exhibit the following properties: 1) those smaller than albumin exhibit reduced vascular resident times; 2) those approaching albumin in molecular weight (66,500) have the greatest vascular residence times; and those greater than 66,500 shows reduced vascular residence. For example, prothrombin has a half life in circulation of 1.7 days (Halton, M. W., J. Lab. Clin. Med. 1995: Vol. 126-521-529 Recombinant rabbit Albumin fragments containing sequences 1-185, 1-377, and 378-584 were excreted rapidly in the urine and possess plasma life's of only 0.71 days (Sheffield, et., Thrombosis Research 99, 613-621 (2000). Recently, work by the same group has verified that disulphide-linked albumin dimers are cleared more rapidly than the parent molecule having a ½ life reduced by approximately 60% that of the monomer (McCurdy et al., J. Lab. Clin. Med. (2004) 143(2) 115-124). Although, surface charge has been implicated as one of the active processes in the retention of plasma proteins, clearly molecular weight is playing a predominant role with regard to albumin. In the practice of this invention and in order to obtain therapeutic proteins and other agents with the desired vascular residence times, peak vascular residence times should be achieved for those fusion proteins with molecular weights approaching albumin. In this case to obtain a maximum extension of the ½ life for a therapeutic protein of 20,000 MW would require the construction of a recombinant fusion with an albumin fragment with a molecular weight of approximately 45,000. Molecular weights greater than albumin will again reduce the ½ life. Recombinant albumin fusion products which involve polymers of albumin or polymers of albumin fragments can therefore also be used to tune the vascular residence times of therapeutic agents. Variations from this rule would be expected to occur on a case by case basis depending on among other factors, glycosylation of the therapeutic portion of the fusion product, or other factors, which induce catabolism and rapid clearance. Consequently, examining the vasculature residence times of various fragments when attempted to assess which fragment will result in an optimal fusion polypeptide can also be applied to obtained the desired result.

What is claimed is:

1. A fusion polypeptide comprising a fragment of human serum albumin containing at least one domain or subdomain or combinations thereof and a therapeutically active polypeptide attached thereto in such a manner wherein the human serum albumin fragment optimizes the half-life of said therapeutically active polypeptide in the bloodstream depending on the molecular weight of the fragment.

2. The fusion polypeptide according to claim 1 wherein the albumin domain or subdomain is selected from the group consisting of domains I, II, III, I-II, II-III, IB-II, I-IIA and subdomains IA-IB-IIA and IB-IIA-IIB.

3. The fusion polypeptide according to claim 1 wherein the therapeutically active polypeptide is of human origin.

4. The fusion polypeptide according to claim 1, wherein said therapeutically active polypeptide comprises all or part of a polypeptide selected from the group consisting of enzymes, enzyme inhibitors, antigens, antibodies, hormones, coagulation factors, interferons, cytokines, growth factors, differentiation factors, factors involved in the genesis of bone tissues, factors involved in the resorption of bone factors, chemotactic factors, cell motility factors, migration factors, cytostatic factors, bactericidal factors, antifungal factors, plasma adhesive molecules, interstitial adhesive molecules and extracellular matrices.

5. The fusion polypeptide according to claim 1 that is prepared using recombinant means

6. The fusion polypeptide according to claim 1 wherein the half-life of the therapeutic polypeptide is extended.

7. The fusion polypeptide according to claim 1 wherein the half-life of the therapeutic polypeptide is reduced.

8. An isolated nucleic-acid encoding the fusion polypeptide according to claim 1.

9. The nucleotide acid according to claim 8, further comprising a leader sequence permitting the secretion of the expressed polypeptide.

10. An expression cassette comprising the nucleotide sequence according to claim 8 under the control of a promoter region.

11. A recombinant cell comprising the nucleotide sequence according to claim 8.

12. A recombinant cell according to claim 11, which is a yeast, an animal cell, a fungus or a bacterium.

13. A process for preparing a fusion polypeptide comprising a fragment of human serum albumin containing at least one domain or subdomain or combinations thereof and a therapeutically active polypeptide attached thereto in such a manner wherein the human serum albumin fragment optimizes the half-life of said therapeutically active polypeptide in the bloodstream depending on the molecular weight of the fragment, said process comprising culturing the recombinant cell according to claim 12 under conditions which promote said expression, and recovering the polypeptide produced thereby.

14. A pharmaceutical composition comprising the fusion polypeptide according to claim 1 and a pharmaceutically acceptable vehicle, carrier or excipient.

15. The composition according to claim 14 which is suitable for parenteral, oral, intranasal, subcutaneous, aerosolized or intravenous administration in a human or animal.

16. A vaccine comprising an immunogenic amount of the fusion polypeptide according to claim 1.

17. A method of preparing a vaccine comprising fusing to a fragment of human serum albumin containing at least one domain or subdomain or combinations thereof an immunogenic polypeptide in such a manner wherein the human serum albumin fragment optimizes the half-life of said immunogenic polypeptide in the bloodstream depending on the molecular weight of the fragment

18. A method of eliciting an immunogenic reaction in a human or animal comprising administering to said human or animal an immunologically effective amount of the fusion polypeptide according to claim 1.

19. A method of optimizing the half-life of a therapeutic polypeptide when internally administered to humans or animals comprising forming a fusion polypeptide between said therapeutic polypeptide and a fragment of human serum albumin containing at least one domain or subdomain or combinations thereof by attaching said albumin fragment to said therapeutically active polypeptide in such a manner wherein the human serum albumin fragment optimizes the half-life of said therapeutically active polypeptide in the bloodstream depending on the molecular weight of the fragment

20. A method according to claim 19 wherein the half-life of the therapeutic polypeptide is extended.

21. A method according to claim 19 wherein the half-life of the therapeutic polypeptide is reduced.

22. A fusion polypeptide comprising a polymer of human serum albumin and a therapeutically active polypeptide attached thereto in such a manner wherein the human serum albumin polymer optimizes the half-life of said therapeutically active polypeptide in the bloodstream depending on the molecular weight of the polymer.

23. The fusion polypeptide of claim 22 wherein the polymer comprises 2 to 5 albumin monomers.

24. The fusion polypeptide of claim 22 wherein the polymer of human serum albumin is selected from the group consisting of dimers and trimers.

25. A method of optimizing the half-life of a therapeutic polypeptide when internally administered to humans or animals comprising forming a fusion polypeptide between said therapeutic polypeptide and a polymer of human serum albumin in such a manner wherein the human serum albumin polymer optimizes the half-life of said therapeutically active polypeptide in the bloodstream depending on the molecular weight of the polymer
